(19) United States
(12) Patent Application Publication
(10) Pub. No.: US 2025/0114089 A1
(43) Pub. Date: Apr. 10, 2025

(54) MANUFACTURING DEVICE FOR FORMING BARBS ON SUTURES

(71) Applicant: HYUNDAE MEDITECH Co., Ltd., Wonju-si (KR)

(72) Inventors: Mi Hee SONG, Wonju-si (KR); Chul PARK, Yongin-si (KR)

(73) Assignee: HYUNDAE MEDITECH Co., Ltd., Wonju-si (KR)

(21) Appl. No.: 18/530,875

(22) Filed: Dec. 6, 2023

(30) Foreign Application Priority Data
Oct. 10, 2023 (KR) 10-2023-0134236

(51) Int. Cl. A61B 17/06; A61B 17/00

(52) U.S. Cl. CPC A61B 17/06166; A61B 2017/00526; A61B 2017/06176

(57) ABSTRACT

The present disclosure provides a manufacturing device for forming barbs on a suture, the manufacturing device including: a suture supply configured to supply a suture; a distal end mover configured to grip a distal end of the supplied suture and move the suture; a first mold part positioned between the suture supply and the distal end mover and having a first barb formation groove for forming a barb, a suture accommodation groove for accommodating the suture, and a presser passage hole configured to press the suture which are each formed in a symmetric shape in first molds at both sides; a presser coupled to the presser passage hole of the first mold part to press the suture toward the first barb formation groove; a cutter configured to cut a tip of the suture on which formation of the barb is completed; and a suture collector configured to collect the cut suture.

### CROSS-REFERENCE TO RELATED APPLICATION

This application claims priority to and the benefit of Korean Patent Application No. 2023-0134236, filed on Oct. 10, 2023, the disclosure of which is incorporated herein by reference in its entirety.

### BACKGROUND

### 1. Field of the Invention

The present disclosure relates to a manufacturing device for forming barbs on sutures, and more particularly, to a manufacturing device for forming barbs on a suture by pressing the suture in a mold to manufacture a suture having barbs formed thereon among sutures used for medical or cosmetic purposes.

### 2. Discussion of Related Art

In recent years, a medical suture used to suture a laceration of the skin or an organ has also been widely used for cosmetic purposes to lift sagging skin or improve wrinkles.

Conventionally, a suture made of a material such as polydioxanone (PDO) that dissolves in the skin has been inserted subcutaneously to induce collagen formation, but nowadays, a suture having barbs formed on a surface thereof is used to pull the skin or improve wrinkles.

Barbs are also referred to as protrusions or cogs.

In order to form barbs on a surface of a suture, a method of partially cutting out the surface of the suture obliquely using a blade is mostly used, which has problems in that, not only is a cross-section of the suture reduced, but also stress is concentrated on cut-out portions, which makes the suture prone to breakage.

In order to address such problems, methods of forming barbs on a surface of a suture by inserting the suture into a mold and heating and pressing the suture have been proposed.

When barbs are formed using molds, since the barbs should be formed along a boundary surface between two molds facing each other, it is difficult to form barbs (2) along a circumference of a suture (1) as in FIG. 1. Korean Patent Registration No. 10-1688020 discloses forming barbs in various directions using a mold, but from the drawing for describing that embodiment, it seems that only barbs symmetrical to each other in both directions can be formed.

In molds of the related art disclosed in FIG. 2, a suture (1) is inserted into a mold (10), and then a plurality of protruding portions (21) of a sliding mold (20) are pushed into a plurality of mold grooves (11) in the mold to form barbs. In such a case, in consideration of a direction of insertion of the sliding mold (20), it is not possible to simultaneously form barbs with distal ends formed in opposite directions. Also, since it is generally not possible to form barbs in various directions using molds formed as a pair, directions of barbs should be controlled by combining multiple molds, but for an object with a small diameter such as a suture, there is a problem in that it is very difficult to manufacture molds.

In particular, in the formation of barbs using the conventional molds, intervals between the barbs, directions that sharp ends of the barbs face in a cross-sectional view of the suture, the number of barbs, and the like are determined by the molds, and thus there is a problem in that it is not possible to form barbs in various shapes.

### RELATED ART DOCUMENTS

### Patent Documents

KR 10-1921980 B1, Nov. 27, 2018, FIG. 2
KR 10-1688020 B1, Dec. 21, 2016, FIG. 3, FIG. 5

### SUMMARY OF THE INVENTION

The present disclosure is for forming barbs by compression molding using molds without partially cutting out a suture and is directed to providing a manufacturing device that allows intervals between barbs, directions that sharp ends of the barbs face, and the number of barbs to be controlled arbitrarily and at a low cost.

The present disclosure provides a manufacturing device for forming barbs on a suture, the manufacturing device including: a suture supply configured to supply a suture; a distal end mover configured to grip a distal end of the supplied suture and move the suture; a first mold part positioned between the suture supply and the distal end mover and having a first barb formation groove for forming a barb, a suture accommodation groove for accommodating the suture, and a presser passage hole configured to press the suture which are each formed in a symmetric shape in first molds at both sides; a presser coupled to the presser passage hole of the first mold part to press the suture toward the first barb formation groove; a cutter configured to cut a tip of the suture on which formation of the barb is completed; and a suture collector configured to collect the cut suture.

The manufacturing device may further include a rotator positioned between the suture supply and the first mold part and configured to cause the suture to be twisted.

The manufacturing device may further include: a second mold part positioned between the suture supply and the distal end mover and having a second barb formation groove for forming a barb in a direction different from a direction that a sharp end of the first barb formation groove faces, a suture accommodation groove for accommodating the suture, and a presser passage hole configured to press the suture which are each formed in a symmetric shape in second molds at both sides; and a presser coupled to the presser passage hole of the second mold part to press the suture toward the second barb formation groove.

The first mold part or the second mold part, and the presser coupled thereto may be provided as a plurality of first mold parts or a plurality of second mold parts, and a plurality of pressers.

In a case in which the first mold part or the second mold part is provided as the plurality of first mold parts or the plurality of second mold parts, with respect to a longitudinal direction of the suture as an axis, directions that distal ends of barbs face may be formed to be different from each other in each mold part constituting the first mold parts or the second mold parts.

A device configured to heat each mold part may be further provided in the first mold part or the second mold part.

### BRIEF DESCRIPTION OF THE DRAWINGS

The above and other objects, features and advantages of the present disclosure will become more apparent to those of ordinary skill in the art by describing exemplary embodiments thereof in detail with reference to the accompanying drawings, in which:

FIG. 1 is a view illustrating a form in which barbs are formed on a suture;

FIG. 2 is a view illustrating an example of conventional molds for manufacturing a suture having barbs formed thereon;

FIG. 3 is a view illustrating a manufacturing device according to one embodiment of the present disclosure;

FIGS. 4 to 6 are views for describing an operational relationship of the manufacturing device according to one embodiment of the present disclosure;

FIG. 7 is a perspective view illustrating a first mold part and a presser;

FIG. 8 is a perspective view illustrating the first mold part and the presser at one side that constitute the first mold part;

FIG. 9 is a view illustrating cross-sections and an arrangement form of first and second molds according to another embodiment of the present disclosure;

FIG. 10 is a view illustrating the shapes of barbs that may be formed according to the arrangement form of the first and second molds; and

FIGS. 11 and 12 are views illustrating the first mold part and the presser according to still another embodiment of the present disclosure.

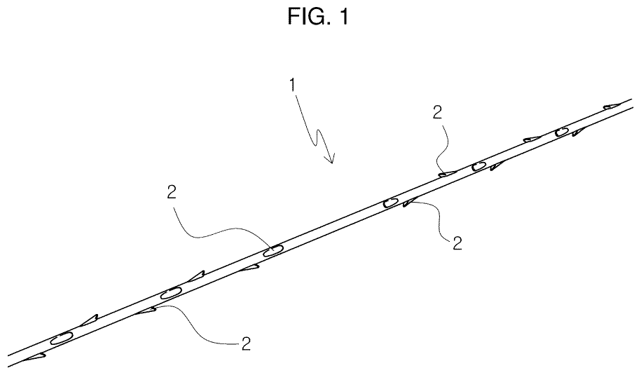

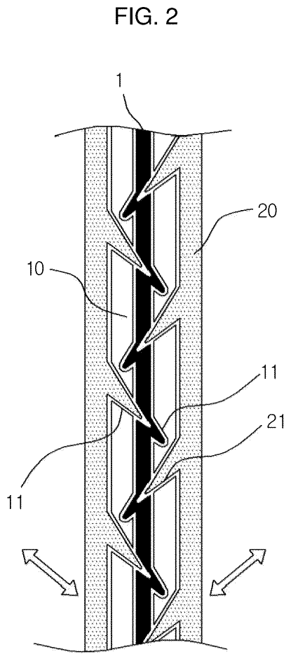

### DETAILED DESCRIPTION OF EXEMPLARY EMBODIMENTS

Hereinafter, the present disclosure will be described in more detail according to embodiments thereof with reference to the accompanying drawings.

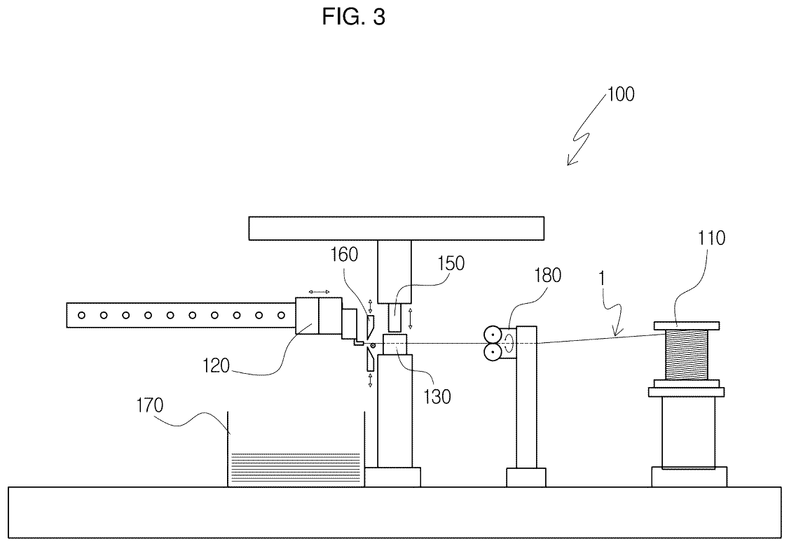

FIG. 3 is a view illustrating a manufacturing device for forming barbs on a suture according to one embodiment of the present disclosure.

A manufacturing device 100 includes a suture supply 110 configured to supply a suture 10, a distal end mover 120 configured to grip a distal end of the supplied suture 1 and move the suture 1, a first mold part 130 positioned between the suture supply 110 and the distal end mover 120 and configured to form barbs on the suture 1, a presser 150 configured to press the suture in the first mold part 130, a cutter 160 configured to cut a tip of the suture on which formation of the barbs is completed, and a suture collector 170 configured to collect the cut suture.

The suture supply 110 has the form of a reel and has the suture 1 wound therearound, and the distal end mover 120 grips the distal end of the suture 10 and moves along a rail.

The suture 1 passes through the first mold part 130 between the suture supply 110 and the distal end mover 120, and the presser 150 presses the suture positioned in first molds 140 of the first mold part 130 to form a barb on the suture.

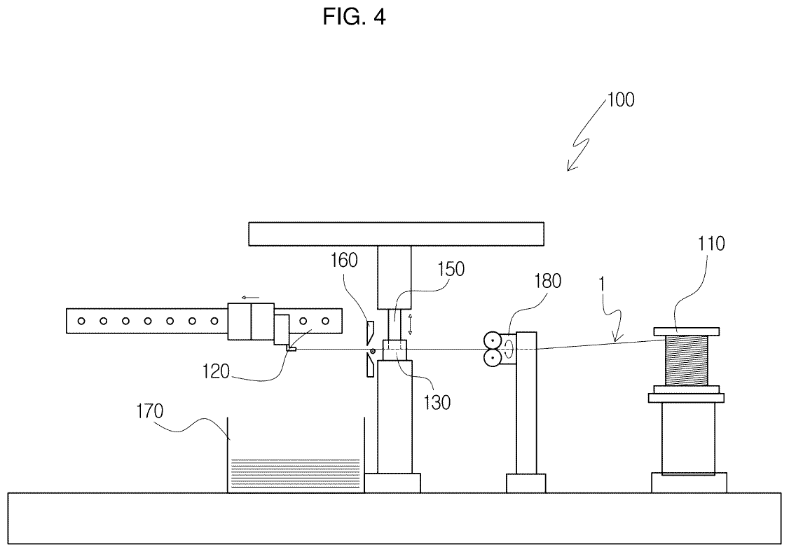

After a pair of molds constituting the first molds 140 are spaced apart from each other, the suture on which the barb is formed by the first mold part 130 and the presser 150 is moved in a distal end direction by the distal end mover 120 to form a subsequent barb thereon, and barbs are sequentially and continuously formed as the pair of molds are coupled to each other again, and the presser 150 presses the suture to form a barb thereon (see FIG. 4).

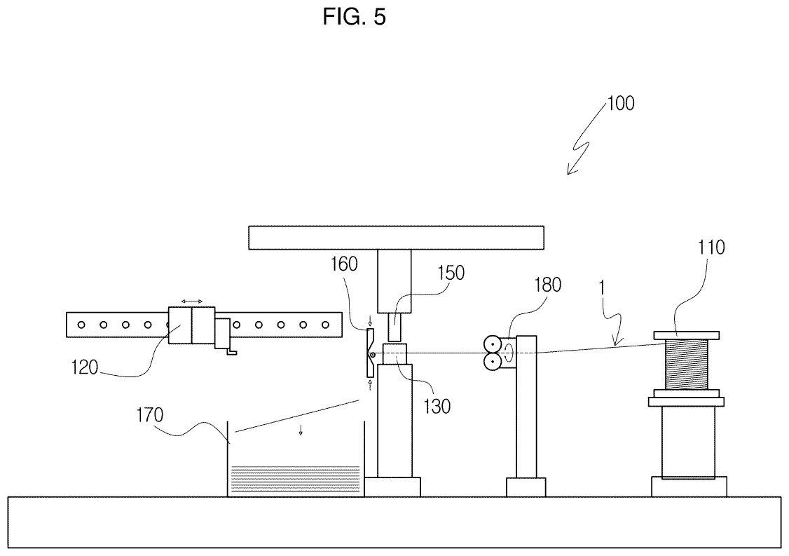

After the barbs are formed on the suture across a predetermined length of the suture, when the suture is cut by the cutter 160, the suture falls onto and is collected by the suture collector 170 (see FIG. 5).

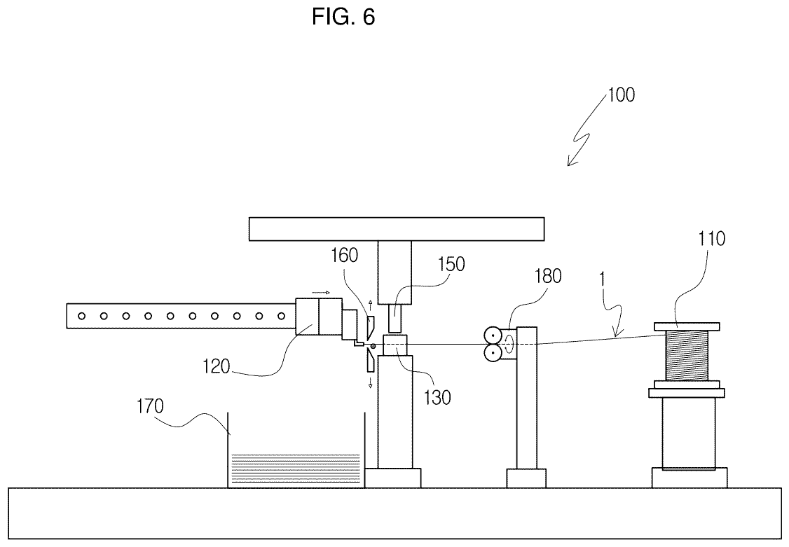

The distal end mover 120 moves toward the cutter 160 again, grips the distal end of the suture, and repeats the process of FIG. 4 (see FIG. 6).

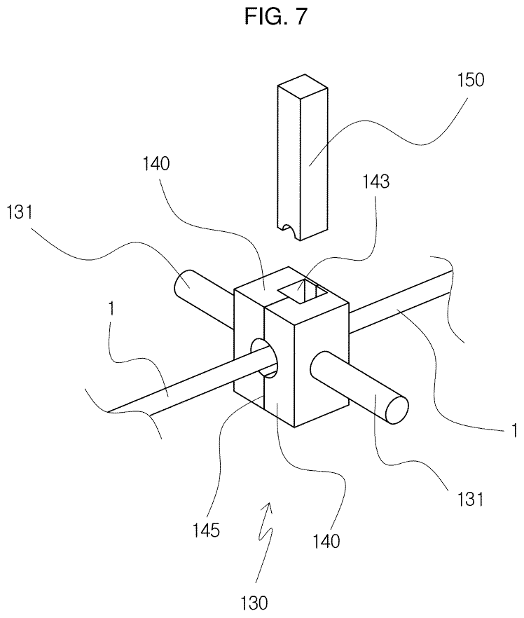

As illustrated in FIG. 7, the first mold part 130 includes the pair of first molds 140 and mold movers 131 configured to move the first molds 140.

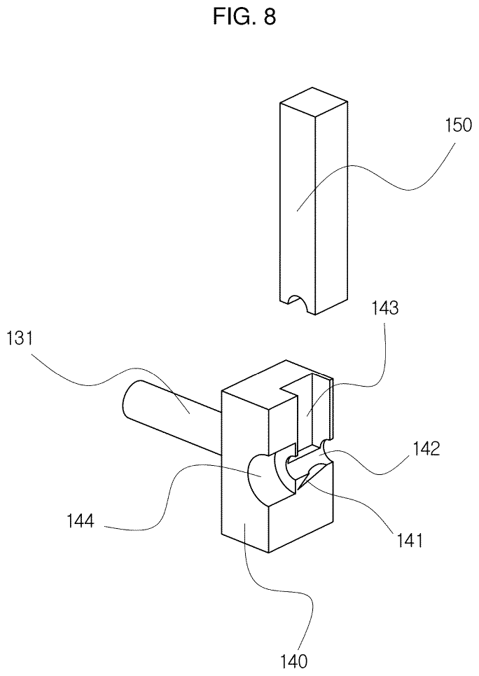

As illustrated in FIG. 8, in the first mold part 130, a first barb formation groove 141 having a shape corresponding to the shape of a barb, a suture accommodation groove 142 in which the suture inserted into the mold is accommodated except for a portion of the suture in the first barb formation groove 141, and a presser passage groove 143 through which the presser 150 pressing the suture toward the first barb formation groove 141 passes are each formed in a symmetric shape by a half portion thereof being formed in each of the pair of first molds 140 at both sides. For example, the suture accommodation groove 142 is a groove having a semicircular cross-section in the first mold 140 at one side and is formed as a groove having a circular cross-section when the first mold 140 at one side is coupled to the first mold at the other side.

The pair of first molds 140 are coupled to or separated from each other by the mold mover 131 provided in each of the pair of first molds 140.

The mold mover 131 may be formed as any of various known devices such as a cylinder or a linear motor.

An avoidance groove 144 configured to prevent the suture from coming in contact with another portion of the first mold 140 while, after a portion of the suture on which a barb is already formed is moved, another barb is being formed thereon may be further provided in the first mold 140. The avoidance groove 144 may be omitted when a width of the first mold 140 is appropriately adjusted.

A distal end groove 151 having a semicircular or arc-shaped cross-section is formed at a distal end of the presser 150, and when the distal end groove 151 of the presser 150 presses the suture, a barb is formed on the suture as the suture is pushed into the first barb formation groove 141 due to plastic deformation of the suture.

The suture is made of a biodegradable polymer material such as polydioxanone (PDO) that is commonly known, and since PDO has a melting point in a range of 110° C. to 115° C. and a glass transition temperature in a range of −12° C. to −5° C., a barb can be formed on the suture by pressing the suture at room temperature (25° C.) without separately heating the suture.

A suture made of a material such as polyglycolic acid (PGA) has a melting point in a range of 220° C. to 230° C. and a glass transition temperature in a range of 40° C. to 50° C., and thus the first mold part for forming a barb on the PGA suture may further include a heater (not illustrated) that can increase the temperature of the molds. The heater includes a heat generator, a temperature sensor, and the like and maintains a certain temperature with a separate controller. Since this is apparent to those of ordinary skill in the art, detailed description thereof will be omitted.

According to the above embodiment, barbs are formed in a straight line on an outer circumferential surface of the suture in an axial direction.

In the next embodiment, forming barbs in a spiral shape on an outer circumferential surface of the suture will be described.

The suture 1 whose one side distal end is fixed by the distal end mover 120 is twisted by a rotator 180 installed between the suture supply 110 and the first mold part 130, and accordingly, a direction that a distal end of the previously formed barb faces and a direction that a distal end of a subsequently formed barb faces may be made different.

That is, when barbs are formed by sequentially twisting the suture by a predetermined angle each time, a plurality of barbs disposed in a spiral shape may be obtained.

The rotator 180 of the present embodiment may cause the suture to be twisted as the suture is fitted between two rollers, and the two rollers rotate the suture in the axial direction.

In the process described with reference to FIGS. 4 to 6, the rotator may selectively rotate in both directions to twist the suture or untwist the twisted suture.

According to still another embodiment of the present disclosure, directions that sharp ends of the barbs formed on the outer circumferential surface of the suture face may be formed to be opposite to each other.

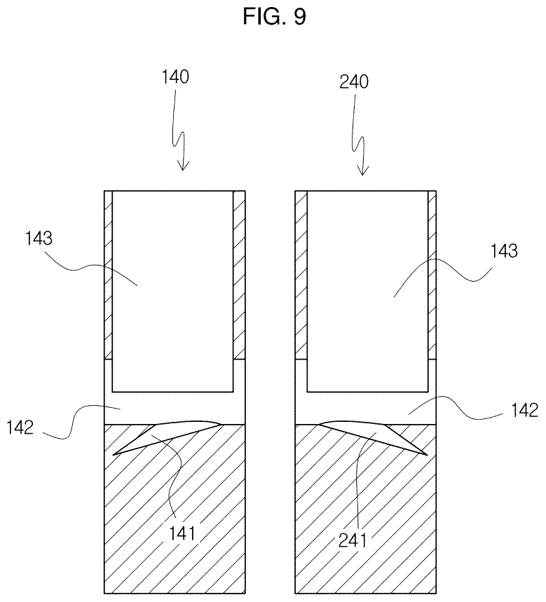

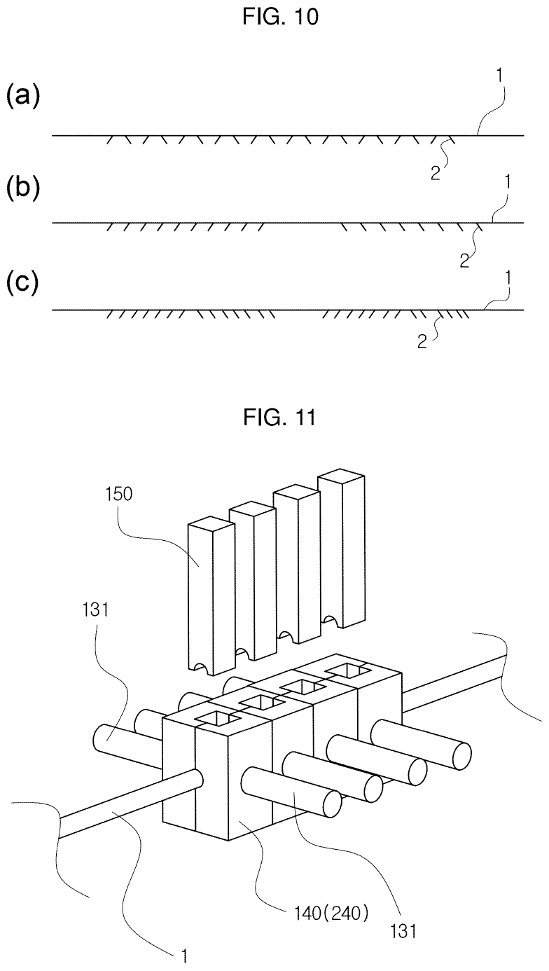

As illustrated in FIG. 9, the manufacturing device 100 may further include a second mold 240 which is different from the first mold 140 in that a second barb formation groove 241 is formed in a direction different from a direction that the sharp end of the first barb formation groove 141 of the first mold 140 faces, and barbs having sharp ends formed toward one side may be formed by the first mold 140, while barbs having sharp ends formed toward the other side are formed by the second mold 240. Accordingly, barbs with sharp ends facing directions opposite to each other may be formed in various shapes as illustrated in FIG. 10. FIG. 10 shows various shapes of barbs that may be formed using the first and second molds 140 and 240 of FIG. 9, but the shapes of barbs that may be formed are not limited thereto.

In the description herein, a mold (part) indicates the first mold (part), the second mold (part), or an arrangement in which the first and second mold (parts) are arbitrarily disposed together according to a certain purpose.

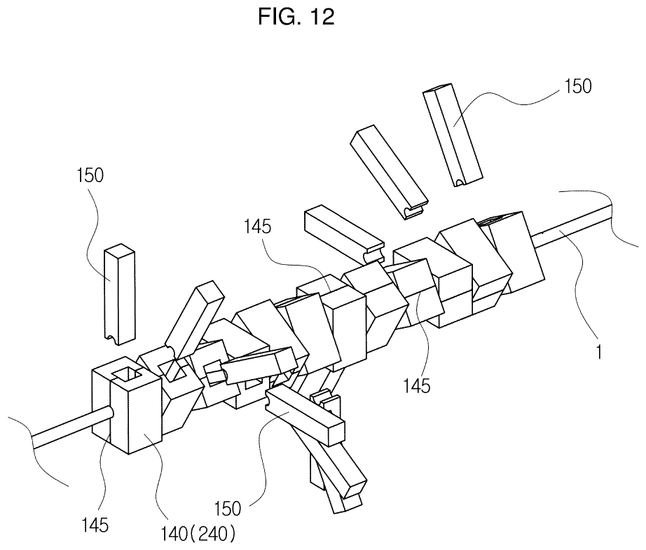

According to yet another embodiment (FIG. 11) of the present disclosure, since the manufacturing device includes a plurality of mold parts, it is possible to simultaneously form a plurality of barbs. In such a case, there is an advantage in that productivity can be increased compared to the above-described embodiments.

Also, when the suture is pressed after being twisted by the rotator 180, barbs may be formed in a spiral shape on the suture.

In order to place barbs in a spiral shape using a plurality of mold parts of the present disclosure without using the rotator 180, as in FIG. 12, angles that joining surfaces 145 of molds of each mold part form with an axis of the suture may be varied to manufacture a suture having barbs formed thereon in a spiral shape in one process. FIG. 12 illustrates a case in which molds and pressers are placed by being rotated 30° each time so that sharp ends of barbs are formed by being rotated 30° each time in a spiral shape, and illustration of mold movers is omitted to help with understanding of the structure.

According to the present disclosure, since barbs can be formed by compression molding using molds without partially cutting out a suture, intervals between barbs, directions that sharp ends of the barbs face, and the number of barbs can be controlled arbitrarily or at a low cost.

The present disclosure has been described in more detail above based on the drawings so that those of ordinary skill in the art can easily practice the present disclosure. All terms including technical or scientific terms used herein have the same meaning as commonly understood by those of ordinary skill in the art to which the present disclosure pertains. Terms, such as those defined in commonly used dictionaries, should be interpreted to have meanings consistent with their meanings in the context of the relevant art and should not to be construed in an idealized or overly formal sense unless expressly so defined herein.

What is claimed is:

1. A manufacturing device for forming barbs on a suture, the manufacturing device comprising:
a suture supply configured to supply a suture;
a distal end mover configured to grip a distal end of the supplied suture and move the suture;
a first mold part positioned between the suture supply and the distal end mover and having a first barb formation groove for forming a barb, a suture accommodation groove for accommodating the suture, and a presser passage hole configured to press the suture which are each formed in a symmetric shape in first molds at both sides;
a presser coupled to the presser passage hole of the first mold part to press the suture toward the first barb formation groove;
a cutter configured to cut a tip of the suture on which formation of the barb is completed; and
a suture collector configured to collect the cut suture.

2. The manufacturing device of claim 1, further comprising a rotator positioned between the suture supply and the first mold part and configured to cause the suture to be twisted.

3. The manufacturing device of claim 1, further comprising:
a second mold part positioned between the suture supply and the distal end mover and having a second barb formation groove for forming a barb in a direction different from a direction that a sharp end of the first barb formation groove faces, a suture accommodation groove for accommodating the suture, and a presser passage hole configured to press the suture which are each formed in a symmetric shape in second molds at both sides; and
a presser coupled to the presser passage hole of the second mold part to press the suture toward the second barb formation groove.

4. The manufacturing device of claim 1, wherein the first mold part and the presser coupled thereto are provided as a plurality of first mold parts and a plurality of pressers.

5. The manufacturing device of claim 4, wherein, in a case in which the first mold part is provided as the plurality of first mold parts, with respect to a longitudinal direction of the suture as an axis, directions that distal ends of barbs face are formed to be different from each other in each mold part constituting the first mold parts.

6. The manufacturing device of claim 1, wherein a device configured to heat each mold part is further provided in the first mold part.

7. The manufacturing device of claim 3, wherein the first mold part or the second mold part, and the presser coupled thereto are provided as a plurality of first mold parts or a plurality of second mold parts, and a plurality of pressers.

8. The manufacturing device of claim 3, wherein a device configured to heat each mold part is further provided in the first mold part or the second mold part.
